(19) United States
(12) Patent Application Publication
(10) Pub. No.: US 2005/0113648 A1
(43) Pub. Date: May 26, 2005

(54) Bidirectional monitoring system capable of a medical diagnosis and a commercial broadcast

(76) Inventors: Soohyun Yang, Seoul (KR); Miheon Jo, Sungnam-shi (KR)

Correspondence Address: MORGAN & FINNEGAN, L.L.P., 3 WORLD FINANCIAL CENTER, NEW YORK, NY, 10281-2101, US

(21) Appl. No.: 10/725,273

(22) Filed: Nov. 24, 2003

(51) Int. Cl. A61B 5/00

(52) U.S. Cl. 600/300; 600/365; 600/549; 600/485; 600/573; 128/903; 128/904; 435/10

(57) ABSTRACT

The disclosed is a bidirectional monitoring system capable of a medical diagnosis and a commercial broadcast, comprising: a main server for providing commercial information, detailed commercial information in case a certain user selects the commercial information, a service page for receiving a user's request for the commercial information, and offering diagnosis result data in response to a medical diagnosis request which is received; a data communication network for allowing many and unspecified persons to access the main server in an on-line manner; and a monitoring device for receiving the commercial information from the main server to display them on a main screen after getting an on-line access to the main server via the data communication network, creating a subsidiary window on upper portion of one side of the main screen to output another commercial information or display medical diagnosis request data input according to the user's handling and then transmit them to the main server, and outputting a page for outputting diagnosis result data corresponding to the medical diagnosis request data.

### FIELD OF THE INVENTION

The present invention relates to a bidirectional monitoring system capable of a medical diagnosis and a commercial broadcast. More specifically, the present invention relates to a bidirectional monitoring system capable of a medical diagnosis and a commercial broadcast, in which a system capable of collecting and analyzing person's urine is connected to a liquid crystal display device so that a certain user can immediately recognize data of her/his urinary analysis as well as be allowed to undergo a medical doctor's diagnosis by transmitting the data of her/his urinary analysis to a medical institution at a long distance in case of abnormal conditions.

### BACKGROUND OF THE INVENTION

Person's urine functions as an important measure which shows person's health conditions and can be used to easily detect abnormal conditions by observing its color, volume, or frequency at ordinary times.

In other words, person's urine functions as a path which absorbs necessary materials and discharges unnecessary materials from person's blood in a kidney and can be used to detect abnormal conditions in person's blood composition, circulatory system, liver, as well as excretory/urinary system. For example, since it is possible to predict a kidney disease, diabetes, diabetes insipidus, pyclitis, prostatic hypertrophy, pituitary tumor, etc., just by a urine test. Therefore, it has been indispensable in a basic medical test.

For this reason, the majority of medical institutions offer the urinary analysis in such a way that a cup is distributed to a user, the user's urine is taken in the cup, and then a part of the urine is input to the analyzer.

As a consequence, in places where require a frequent health check such as old people's residences, the aforementioned method of the urine test was cumbersome and consumed a lot of times to collect the urine.

### SUMMARY OF THE INVENTION

Accordingly, the present invention is contrived to solve the cumbersome problems, and an object of the present invention is to provide a bidirectional monitoring system capable of a medical diagnosis and a commercial broadcast, in which users can be provided with commercial information and give an order for that, as well as check their health conditions with easy control in places such as their own homes.

Another object of the present invention is to provide a bidirectional monitoring system capable of a medical diagnosis and a commercial broadcast, in which health check data such as levels of blood glucose, blood pressure, and body temperature measured by using a blood glucose tester, a blood pressure tester, a clinical thermometer, and the like are transmitted to a monitoring system and converted into a graphical format relevant to a reference graph related with normal conditions to display them on the same screen in an overlapping manner so that a self-diagnosis can be easily achieved.

Still another object of the present invention is to provide a bidirectional monitoring system capable of a medical diagnosis and a commercial broadcast, in which a system capable of collecting and analyzing person's urine is connected to a liquid crystal display device so that a certain user can immediately recognize data of her/his urinary analysis as well as be allowed to undergo a medical doctor's diagnosis by transmitting the data of urinary analysis to a medical institution at a long distance in case of abnormal conditions.

A further object of the present invention is to provide a bidirectional monitoring system capable of a medical diagnosis and a commercial broadcast which allows users to easily recognize the results of her/his urine test by displaying them on a screen in a graphical illustration.

An additional object of the present invention is to provide a bidirectional monitoring system capable of a medical diagnosis and a commercial broadcast, in which the results of the urine test is transmitted to a medical institution at a long distance in a voice message type or a literal message type.

In order to achieve the above objects, according to the present invention, a bidirectional monitoring system capable of a medical diagnosis and a commercial broadcast comprises: a main server for providing commercial information, detailed commercial information in case a certain user selects the commercial information, a service page for receiving a user's request for the commercial information, and offering diagnosis result data in response to a medical diagnosis request which is received; a data communication network for allowing many and unspecified persons to access the main server in an on-line manner; and a monitoring device for receiving the commercial information from the main server to display them on a main screen after getting an on-line access to the main server via the data communication network, creating a subsidiary window on upper portion of one side of the main screen to output another commercial information or display medical diagnosis request data input according to the user's handling and then transmit them to the main server, and outputting a page for outputting diagnosis result data corresponding to the medical diagnosis request data.

In addition, according to the present invention, the bidirectional monitoring system further comprises a medical institution server, and the monitoring device includes a connection terminal which can be connected to a health check device, whereby the monitoring device determines if the health check data are input from the health check device when the health check device is connected to the connection terminal, transmits the health check data to the medical institution server in case the health check data are input, compares the health check data with health reference data registered previously so as to output user's health result data recognizable from the health check data on the main screen or the subsidiary window in a graphical illustration, compares the health check data with transmission limitation data registered previously so as to transmit the health check data to the main server or the medical institution server in case the health check data excess the transmission limitation data.

The health check device includes a blood glucose tester, a blood pressure tester, or a clinical thermometer.

In the main server, the commercial information contains link information which enables an on-line connection to a corresponding sponsor's terminal so that an ordering data corresponding to the commercial information can be input by way of an output screen of the commercial information and transmitted.

On the other hand, the bidirectional monitoring system further comprises a bidet including a pressure sensor.

In addition, the bidet is connected to the monitoring system having functions of a medical diagnosis and a communication, and the monitoring device is turned on by the pressure sensor in the bidet in case a user sits down on the bidet to accomplish the functions of a medical diagnosis and a communication.

Furthermore, the monitoring system further comprises an analysis means for collecting user's urine and analyzing the user's urinary composition, and the monitoring device analyzes the user's health conditions by using the user's urinary composition input from the analysis means, outputs analysis results in a graphical illustration, compares the analysis results with transmission limitation data registered previously, and transmit the analysis results by using data of the user's terminal registered previously in case the analysis results excess the transmission limitation data.

Moreover, the monitoring device comprises: a storage unit for storing user's personal data, mobile terminal numbers corresponding to the user's personal data, and at least one of transmission limitation data and reference data for the urinary composition; a control unit for outputting the urinary composition data input from the analysis means in a graphical illustration to compare them with the reference data, simultaneously outputting analysis results recognizable from the graphical illustration, and transmitting analysis result data to a corresponding mobile phone to notify it of a recipient in case any of the analysis result data excess the transmission limitation data; a display unit for displaying an analysis graph and the analysis results on a screen to show them to the user in response to controlling of the control unit; and a transmit unit for transmitting the analysis result data to a corresponding mobile phone in response to controlling of the control unit.

In addition, the monitoring device further comprises a tuner capable of receiving terrestrial broadcast signals. In this case, the control unit controls the tuner according to predetermined conditions so that broadcast signals of a predetermined channel can be received and displayed on the display unit.

In addition, the monitoring device further comprises a receive device capable of receiving cable broadcast signals. In this case, the control unit controls the receive device according to predetermined conditions so that broadcast signals of a predetermined channel can be received and displayed on the display unit.

Particularly, the control unit performs controlling in such a way that the screen on the display unit can be split into a plurality of windows and different image signals are output to the split windows. The broadcast signals output to the split window correspond to commercial broadcast signals.

In addition, the monitoring device accumulates/stores the analysis results input from the analysis means according to respective users and then transmits them on a regular or irregular basis by using data of user's terminals registered according to users. In this case, data of the user's terminals registered previously in the monitoring device are stored in relation with respective user's personal data and correspond to terminal numbers for connection to the doctor in charge.

In addition, the monitoring device further comprises a personal identification data input unit through which a certain user can input personal data, and the personal identification data input unit includes a key panel, a card reader, or a fingerprint detector.

In addition, the analysis means comprises: a urine detect sensor for sampling a small amount of user's urine to detect it; an analyzer for absorbing a small amount of the urine from the urine detect sensor and analyzing its urinary composition; and a communication interface unit for converting the urinary composition analyzed by the analyzer into urinary composition data to transmit them to a liquid crystal display means.

### BRIEF DESCRIPTION OF THE DRAWINS

The above and other objects, advantages and features of the present invention will become apparent from the following description of preferred embodiments given in conjunction with the accompanying drawings, in which:

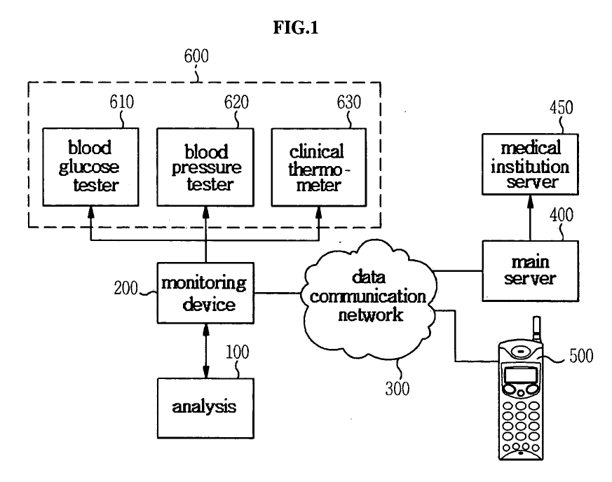

FIG. 1 is a block diagram for explaining organization of the bidirectional monitoring system capable of a medical diagnosis and a commercial broadcast according to the present invention;

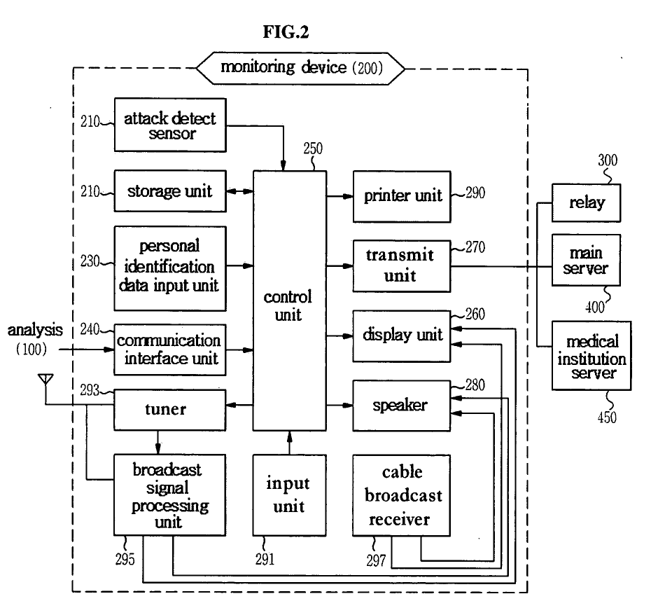

FIG. 2 is a block diagram for explaining organization of the monitoring device shown in FIG. 1;

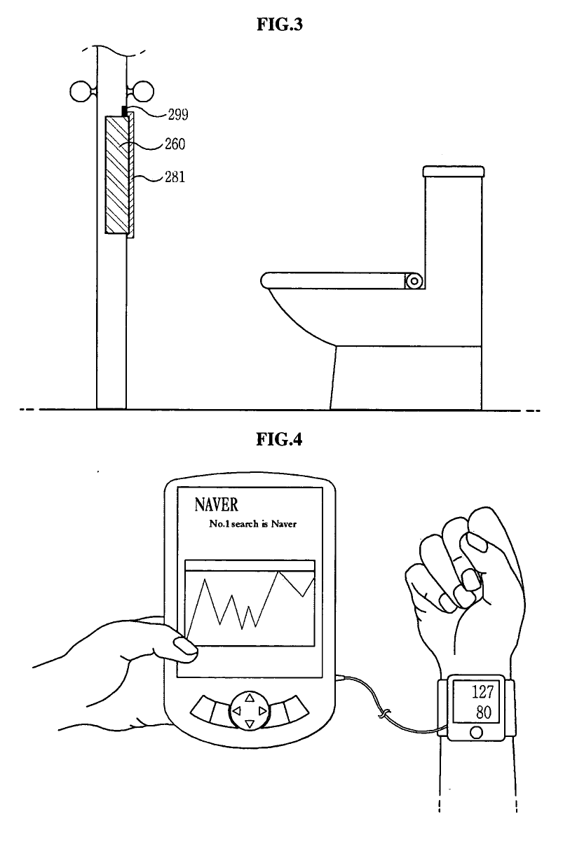

FIGS. 3 to 5 are schematic diagrams for explaining preferred embodiments according to the present invention;

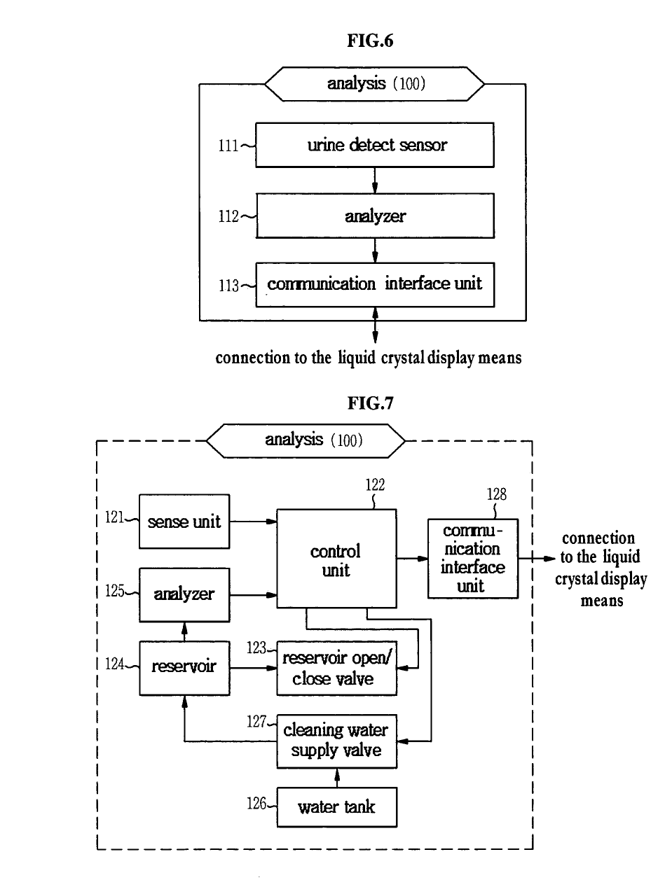

FIG. 6 is a block diagram for explaining organization of the analysis means shown in FIG. 1;

FIG. 7 is a block diagram illustrating another embodiment of FIG. 6; and

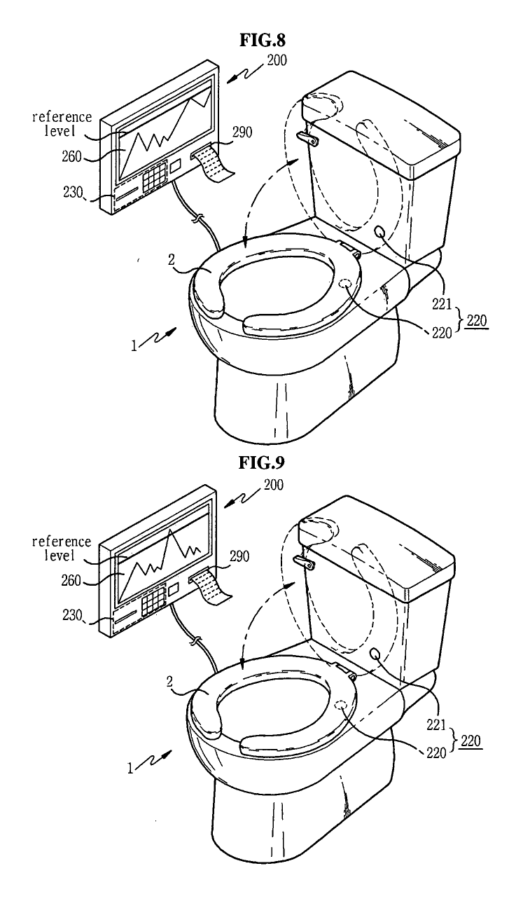

FIGS. 8 and 9 are perspective views illustrating another preferred embodiment of the present invention.

### DETAILED DESCRIPTION OF THE PREFERRED EMBODIMENTS

Now, the preferred embodiments according to the present invention will be described in details with reference to the accompanying drawings.

FIG. 1 is a block diagram for explaining organization of the bidirectional monitoring system capable of a medical diagnosis and a commercial broadcast according to the present invention, FIG. 2 is a block diagram for explaining organization of the monitoring device shown in FIG. 1, FIGS. 3 to 5 are schematic diagrams for explaining preferred embodiments according to the present invention, and FIG. 6 is a block diagram for explaining organization of the analysis means shown in FIG. 1.

As shown in FIG. 1, the bidirectional monitoring system according to the present invention comprises: a main server 400 for providing commercial information, detailed commercial information for a certain user's selection, a service page for accepting user's request for the commercial information, and diagnosis result data in response to the received request for a medical diagnosis; a data communication network 300 for allowing many and unspecified persons to access the main server in an on-line manner; a monitoring device 200 for obtaining an on-line access to the main server 400 via the data communication network 300, receiving the commercial information provided by the main server 400, and outputting them to a main screen, creating a subsidiary window on an upper portion on one side of the main screen to output another commercial information or displaying data of the request for a medial diagnosis which are input according to the user's handling, transmitting the request for a medical diagnosis to the main server 400, and outputting a page for outputting the diagnosis result data corresponding to data of the request for a medical diagnosis; and a medical institution server 500.

The monitoring device 200 further comprises a connection terminal which can be connected to a health check device 600. When the health check device is connected to the connection terminal, the monitoring device determines if the health check data are input from the health check device, transmits the health check data to the medical institution server in case the health check data are input, compares the health check data with health reference data registered previously so as to output user's health result data recognizable from the health check data on the main screen or the subsidiary window in a graphical illustration, compares the health check data with transmission limitation data registered previously so as to transmit the health check data to the main server 500 or the medical institution server in case the health check data excess the transmission limitation data.

The health check device 600 includes a blood glucose tester 610, a blood pressure tester 620, a clinical thermometer 630, and the like.

In the main server 400, the commercial information contains link information which enables an on-line connection to a corresponding sponsor's terminal so that an ordering data corresponding to the commercial information can be input through an output screen of the commercial information and transmitted.

The monitoring device 200 comprises: a storage unit 210 for storing user's personal data, mobile terminal numbers corresponding to the user's personal data, and at least one of transmission limitation data and reference data for the urinary composition; a control unit 250 for outputting urinary composition data input from the analysis means 100 in a graphical illustration to compare them with the reference data, simultaneously outputting analysis results recognizable from the graphical illustration, and transmitting analysis result data to a corresponding mobile phone to notify it of a recipient in case the analysis result data excess the transmission reference data; a display unit 260 for displaying an analysis graph and the analysis results on a screen to show them to the user in response to controlling of the control unit 250; and a transmit unit 270 for transmitting the analysis result data to a corresponding mobile phone in response to controlling of the control unit.

In addition, the monitoring device 200 further comprises: a tuner 293 capable of receiving terrestrial broadcast signals; an input unit 291 having channel selection buttons for outputting channel selection signals in response to the user's request; and a broadcast signal processing unit 295 for accomplishing a signal processing in order to convert the broadcast signals received through the tuner 293 into image signals and audio signals which can be output through the display unit 260 and the speaker 280. At this point, the control unit 250 controls the tuner 293 in response to user's input signals (e.g., channel selection signals) from the input unit 291 so that the broadcast signals of the channel selected by the users can be received or the broadcast signals of a predetermined channel can be received by controlling the tuner 293 according to predetermined conditions, whereby they can be displayed and outputted through the display unit 260 and the speaker 280. In this case, the input unit includes a remote controller, a key pad, and the like.

In addition, the monitoring device 200 further comprises a receive unit 297 capable of receiving cable broadcast signals. At this point, the control unit 250 controls the receive unit 297 according to predetermined conditions so that the broadcast signals of a predetermined channel can be received and corresponding images can be displayed on the display unit 260.

In addition, the screen of the display unit 260 is controlled to be split into a plurality of windows by the control unit 250 so that different image signals can be output to the respective split windows. Also, the broadcast signals output to the split windows may be commercial broadcast signals.

The monitoring device 200 further comprises a protective plate 281 for protecting the display unit 260.

The monitoring device 200 further comprises an attack detect sensor 299 for sensing an external attack and outputting an attack detect signal. At this point, the control unit 250 performs controlling in such a way that an alarm sound can be output through the speaker in response to the attack detect signal from the attack detect sensor 299.

The monitoring device 200 accumulates/stores the analysis results input from the analysis means 100 according to users and then transmits them on a regular or irregular basis by using data of user's terminals registered according to users. At this point, data of the user's terminals registered previously in the monitoring device are stored in relation with the respective user's personal data and may be the terminal numbers for connections to the doctors in charge of the respective users.

The transmit unit 270 transmits the analysis results to a recipient by means of a short message service.

The monitoring device 200 further comprises a personal identification data input unit 230 by which a certain user can input her/his personal data. The personal identification data input unit 230 includes a key panel, a card reader, a fingerprint detector, and the like.

Particularly, a communication interface unit 240 of the monitoring device 200 is constructed to communicate with the analyzer 100 and the health check device 600 in order to receive the health check data from the analyzer 100 and the health check device 600.

Also, the personal identification data input unit 230 functions as a means for inputting personal identification data by the user who wants to offer her/his levels of blood glucose, blood pressure, or body temperature or experience the urine test. The personal identification data input unit 230 can be implemented by a variety of means such as a key input device capable of inputting numbers or characters, a fingerprint detector capable of detect user's fingerprint, or a card reader capable of reading a card which contains user's identification data. At this point, the card may be a card containing user's personal data provided by the main server 400 or a patient identification card provided by a certain medical institution.

In this case, the transmit unit 270 transmits the analysis results to a recipient by means of a short message service.

As shown in FIG. 6, the analysis means 100 comprises a urine detect sensor 111 for sampling a small amount of user's urine to detect it; an analyzer 112 for absorbing a small amount of urine from the urine detect sensor 111 and performing an analysis by using a program for analyzing urinary composition; and a communication interface unit 113 for converting the urinary composition analyzed by the analyzer 112 into urinary composition data to transmit them to the monitoring device 200.

The analysis means 100 further comprises a urine test paper or a urine sensor 111 in the lower end of the analyzer for absorbing a small amount of the user's urine. The urine test paper can be replaced with new one once it is used, whereas the urine sensor can be replaced with new one after a predetermined period of usage.

As shown in FIG. 7, according to another embodiment of the present invention, an analysis means 100 comprises a sense unit 121 for sensing when a user urinates and outputting a urination sense signal; a control unit 122 for controlling the whole system in such a way that the user's urine can be analyzed in response to the urination sense signal; a reservoir open/close valve 123 for closing a reservoir in response to controlling of the control unit 122 at the initial time of the urinary analysis to charge the reservoir with a certain amount of the user's urine and opening the reservoir in response to the controlling of the control unit 122 at the end of the urinary analysis to discharge the user's urine; a cleaning water supply valve 127 for injecting cleaning water in a water tank into the inside of the reservoir where the urine has been discharged in response to controlling of the control unit 122 to wash urinary remnants out; an analyzer 125 for absorbing a small amount of the user's urine collected in the reservoir 124, analyzing them by using an analysis program, and transmitting analysis results to the control unit 122; and a communication interface unit 128 for converting urinary composition analyzed by the analyzer 125 into urinary composition data and transmitting them to the monitoring device 200.

As shown in FIG. 8, the sense unit 121 includes a first sensor for sensing variation of pressure and outputting a urination sense signal when a user sits down on a water closet 1 and a second sensor 222 for detecting when a user lift up a seat 2 of the water closet 1 and outputting a urination sense signal. In this case, the first sensor corresponds to a pressure sensor and the second sensor corresponds to a magnetic sensor.

In addition, data of the user's terminal 500 registered previously in the monitoring device 200 are stored in relation with corresponding user's personal data and may be a mobile terminal number for transmitting the results of the urinary analysis for a certain user to a doctor in charge or log-in information of a predetermined on-line messenger.

Also, the monitoring device 200 accumulates/stores the analysis results input from the analysis means 100 according to users and then transmits them on a regular or irregular basis by using data of user's terminals registered according to users.

The main server 400 can be implemented on the basis of a WAP as well as an internet so that every user can get an access to it and be provided with predetermined data by using a personal computer or a mobile terminal.

The user's terminal 500 may include the one for the user who requests a urine test or another one for the doctor in charge.

Now, operations of the aforementioned bidirectional monitoring system capable of a medical diagnosis and a commercial broadcast will be described as follows.

### Embodiment 1

The following descriptions are given to the case that the monitoring device 200 according to the present invention is installed in places such as the living room.

First, the monitoring device 200 can be installed in the bed room or the living room as shown in FIG. 3, or manufactured in a portable type as shown in FIG. 4.

As shown in FIGS. 3 and 4, the monitoring device 200 installed in places such as the living room and the bed room functions as an output device for a terrestrial broadcast, a cable broadcast, or a satellite broadcast as well as a device capable of a health check for the users.

In other words, the monitoring system according to the present invention is implemented in such a way that the user can select corresponding functions by using a key pad incorporated in a remote controller or a monitoring device 200.

For example, when a user inputs a request signal for switching to a drive mode for the output device for a terrestrial broadcast through the input unit 291 and a channel selection signal, the control unit 250 controls the tuner unit 293 to receive the terrestrial broadcast signals of the channel that the user has selected and the broadcast signal processing unit 295 converts them into image signals or audio signals appropriate to be output through the display unit 260 and the speaker 280.

Meanwhile, when a user inputs a request signal for switching to a health check mode through the input unit 291 for the purpose of her/his health check, the control unit 250 determines if there is any signal input through the personal identification data input unit 230. In other words, the user should input her/his unique identification data in order to transmit the request signal for a health check to the main server 400 or the medical institution server 450 and receive corresponding diagnosis results. For example, data such as personal number, authentication data distributed by the main server, and patient number distributed by the medical institution registered previously should be transmitted to the main server 400 or the medical institution server 450 together with the health check data.

As described above, when the personal identification data are input through the personal identification data input unit 230, the control unit 250 determines if the health check data are input through the communication interface unit 240 from a predetermined device.

As a result, if the health check data are input, the control unit transmits them together with the personal identification data to the main server 400 or the medical institution server 450 and creates rough health diagnosis results according to predetermined health reference data to output them through the display unit 260 in a graphical illustration.

In this case, the screen on the display unit 260 is split into at least two windows where different image signals are output to them. For example, commercial information can be displayed on the main screen while a typical terrestrial or cable broadcast can be displayed on the subsidiary window.

The commercial information may relate to home shopping malls, or local shops such as restaurants, laundries, and supermarkets in the vicinity of the place where the monitoring device 200 is installed. Also, the commercial information may include an ordering menu. When the ordering menu is selected, an ordering window is presented. Subsequently, when an ordering request is input through the ordering window, ordering data which have been written through the ordering window are transmitted to the terminal of the sponsor who has offered the commercial information so that dealing transactions can be accomplished. Needless to say, a payment section can be included in the ordering window. The ordering menu can be applied to every embodiment of the present invention, and the descriptions for the similar parts will not be given for simplicity.

### Embodiment 2

The following descriptions are given to the case that the monitoring device 200 according to the present invention is installed in places such as public restrooms and the like.

As shown in FIGS. 1 and 3, inside of the restroom door, there are installed the display unit 260 which may be an LCD monitor, the speaker 280 for outputting audios, and the control unit 250 for performing controlling in such a way that the image/voice signals received from a broadcast system at a long distance can be outputted to the display unit 260 and the speaker 280 depending on power on/off and selecting a broadcast channel according to user's channel handling.

In addition, the control unit 250 comprises an alarm interface capable of alarming when there is an attack on the display unit 260 or the speaker 280.

Meanwhile, the door lock device of the restroom where the monitoring device 200 is installed can function as a switch for power on/off the monitoring device. In other words, if the door lock device is in the locked position, the whole system becomes power-on state, and then the control unit 250 performs controlling in such a way that the broadcast signals received from the terrestrial broadcast receiver or the cable broadcast receiver can be displayed on the display unit 260. Otherwise, if the door lock device in the restroom is in the unlocked position, supplied powers are cut off and the image and audio broadcast signals output through the display unit 260 and the speaker 280 are interrupted.

Accordingly, when the user puts the door in the locked position to use the restroom, powers are automatically supplied to the display unit 260 and the speaker 280 to output images and sounds. On the contrary, when the user puts the door in the unlocked position to come out, powers to the display unit 260 and the speaker 280 are automatically cut off to interrupt outputs of the images and sounds.

In this case, the cables for delivering the broadcast signals are wired to pass through the hinge portions in the restroom door. In other words, the hinge portions has a structure that can be used for passages of the communication cables for receiving the terrestrial broadcast signals or the cable broadcast signals. If the image/sound data are transmitted in a wireless manner using over-the-air waves, the hinge portions are not necessary to have cable passage structures. Instead, a tuner for receiving wireless signals can be further included and controlled by the control unit 250.

On the other hand, as shown in FIG. 3, a protective plate 281 is installed on the front surface of the display unit 260 for the purpose of protection and security. The protective plate is made up of a transparent material. In addition, an attack detect sensor 299 is installed in the vicinity of the protective plate 281 to detect an external attack and output an external attack detect signal to the control unit 250. Correspondingly, the control unit 250 outputs an alarm sound through the speaker 280.

In this case, the attack detect sensor 299 can be installed in any portions of the device that is exposed to the external and needs protection and security.

In addition, the screen on the display unit 260 which may be an LCD monitor can be split into at least two windows. The control unit 250 controls the whole system to output different images on the split windows. For example, live sports broadcasts can be displayed on the subsidiary window while advertising films are displayed on the main screen. In this case, it is possible to implement the system in such a way that images are displayed on the screen according to a predetermined broadcast control program or users can arbitrarily select images displayed on respective windows.

Furthermore, an LED panel can be prepared to show some characters such as “in-use” at eye level on the external front surface of the door and also be operated according to the locked/unlocked position of the door lock device. In other words, the LED panel can be turned on to show some characters such as “in-use” when the door lock device is in the locked position, whereas the LED panel can be changed to show no characters or other characters such as “out-of-use” when the door lock device is in the unlocked position.

### Embodiment 3

Following descriptions are given to the case that the monitoring device 200 according to the present invention is applied to a PDA or a TV set.

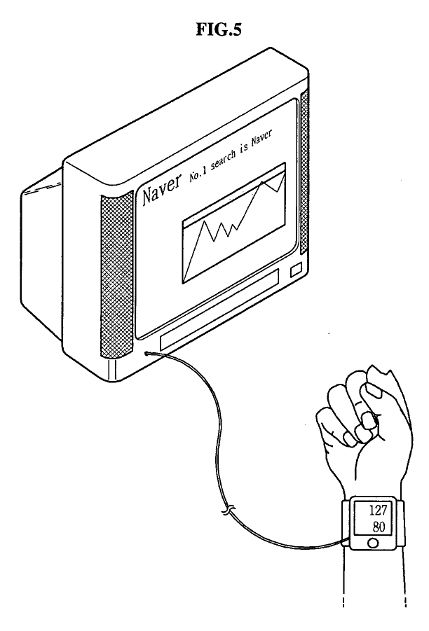

As shown in FIGS. 4 and 5, a health check device is connected to a PDA or a TV set so that the current health conditions of a certain user, e.g., blood pressure, blood glucose, or body temperature, can be measured and then the results of the measurement are converted into numerical data to be output to a communication interface unit (now shown) connected to the communication interface unit 240 installed in a PDA or a TV set.

Subsequently, the control unit 250 allows the numerical data, i.e. health check data, input from the communication interface unit 240 to be transmitted to the main server 400 or the medical institution server 450 and compared with the reference data stored in the storage unit 210 to output the results of comparison on the screen of a PDA or a TV set in a graphical illustration.

In this case, the control unit 250 splits the screen on a PDA or a TV set into at least two windows to show different images on each window. For example, one window can show commercial information, a terrestrial broadcast, a cable broadcast, or a satellite broadcast, while the other window can show the health check results. In other words, each window does not show redundant images of the same source to provide users with a variety of information.

In addition, users can be further provided with a direct ordering menu under the state that the advertising films are displayed. Subsequently, users can be provided with an ordering window which can be used to order the product on advertising when the ordering menu is selected. In other words, the main server 400 is constructed in such a way that the advertising films contain link information which enables an on-line connection to the corresponding sponsor's terminal, whereby the ordering request signal is directly transmitted to the sponsor's terminal by way of a transmit unit 270 according to controlling of the control unit 250 when the user selects the ordering menu.

At this point, the ordering menu can be organized in a manner that the products irrelevant to the advertising films which are being played on the screen can also be ordered. In this case, users should previously register available products which can be irrelevantly ordered and construct a database. Furthermore, the main server 400 should be constructed in such a way that a user can take an access and then give an order directly after a communication network becomes available.

### Embodiment 4

Finally, the following descriptions are given to the case that the monitoring device 200 according to the present invention is connected to a bidet.

First, a user urinates after inputs her/his personal data with guidance from an instructor. Then, the urine sensor 111 samples a small amount of urine and delivers it to the analyzer 112.

In this case, the user can input her/his personal data (i.e., resident registration number) through the key panel in the personal identification data input unit 230. Otherwise, the personal data can be input by using a card containing personal data or a fingerprint method. The method of inputting personal data can be variously implemented considering user's conveniences. Needless to say, personal data such as resident registration numbers and fingerprints should be organized in a database depending on each implementation method.

When the user inputs her/his personal data by way of the personal identification data input unit 230, the control unit 250 receives the user's personal identification data and determines if the user corresponds with the one previously registered on the storage unit 210. As a result, if the user is previously registered, the control unit 250 controls the whole system to perform a urine test.

The control unit 250 receives the urinary composition data by way of the communication interface unit 240. In other words, the analyzer 112 in the analysis means 100 analyzes the urine sampled from the urine detect sensor 111 by using an analysis program and then transmits the urinary composition data to the liquid crystal display means by way of the communication interface unit 113.

Then, the control unit 250 performs controlling in such a way that the urinary composition data from the communication interface unit 240 are output together with the reference level on the screen of the display unit 260 in a graphical illustration as shown in FIGS. 8 and 9 for the purpose of comparison with the reference data stored in the storage unit 210. Simultaneously, the personal identification data from the personal identification data input unit 230 are output on the upper end of the screen of the display unit 260.

Subsequently, the control unit 250 performs controlling in such a way that the analysis results which can be recognized from the graphs are simultaneously output on one end side of the display unit 260 and transmitted to the mobile phone or the registered messenger corresponding to the personal identification data stored in the storage unit 210 by way of the transmit unit 270 to notify it of the recipient in case the analysis result data excess the reference level as shown in FIG. 9.

Meanwhile, the control unit 250 performs controlling in such a way that instruction messages displayed on the display unit 260 can also be provided through the speaker 280 as voice signals to make it easy for users to recognize them. Furthermore, the urinary analysis results can be printed from the printer unit 290 to deposit them.

The urinary analysis result data transmitted through the transmit unit 270 can be delivered to the medical institution server 400 via a data communication network 300. Also, they can be delivered to the user's terminal of the doctor in charge by means of a short message service.

On the other hand, the signals informing the fact that the user's urinary analysis data are received are transmitted from the medical institution server 400 to the terminal of the doctor in charge so that she/he can recognize it by using her/his own terminal.

In addition, the doctor in charge can use her/his terminal 500 to make a diagnosis of the user who has requested a urine test by using the urinary analysis data and then feedback the diagnosis results to the corresponding user. In this case, the diagnosis results may be provided in the type of a voice message or a literal message.

During the urine test or in case the user does not take the urine test, general information is provided on the display unit 260 until the user completes urinating.

According to another embodiment of the analysis means 100, when a user sits down on a water closet or lifts up a seat, the first sensor 221 or the second sensor 222 in the sense unit 121 detects this and outputs a urination sense signal to the control unit 122.

Then, the control unit 122 outputs the urination sense signal to the control unit 250 in the monitoring device 200 by way of the communication interface unit 128, and the control unit 250 outputs instruction messages for a urine test on the display unit 260. For example, messages such as “do you need a urine test?” or “yes/no” can be displayed.

In this case, when a user requests a urine test, the control unit 250 outputs a urinary analysis request signal to the control unit 122 in the analysis means 100 by way of the communication interface unit 240.

Then, the control unit 122 turns on the reservoir open/close valve 123 so that the user's urine can be collected in the reservoir 124, and controls the analyzer 125 so that a urine stick (i.e., a urine test paper) or a urine detect sensor installed in the lower end of the analyzer 125 can absorb a small amount of the user's urine.

The absorbed urine is analyzed by the analysis program in the analyzer 125, and the analyzed urinary composition data are transmitted to the control unit 250 in the monitoring device 200 by way of the communication interface unit 128.

Then, the control unit 122 turns off the reservoir open/close valve 123 so that the urine collected in the reservoir 124 can be discharged, and then turns on the cleaning water supply valve 127 so that the water in the water tank 126 can flow to the injection nozzle (not shown) by way of the cleaning water supply valve 127 and be injected into the reservoir 124 through the injection nozzle to wash the remnants out from the reservoir 124.

As described above, the analysis means 100 according to the present invention has two embodiments, and their before and behind operations are similar to the aforementioned embodiments. Therefore, the redundant descriptions are intentionally omitted.

As described above, according to the present invention, a system capable of collecting and analyzing person's urine is connected to a liquid crystal display device so that a certain user can immediately recognize her/his urine analysis results as well as be allowed to undergo a medical doctor's diagnosis by transmitting her/his urine analysis results to a medical institution at a long distance in case of abnormal conditions. Therefore, users can undergo a health diagnosis by way of a urine test as a basic health test without visiting medical institutions.

In addition, according to the present invention, the urine test results are output on the screen in a graphical illustration so that users can easily recognize the test results.

Furthermore, urine test results are transmitted to a medical institution at a long distance in a voice message type or a literal message type so that a doctor in charge can easily recognize patient's conditions to make a diagnosis

Although the present invention and its advantages have been described in details, it should be understood that the present invention is not limit to the aforementioned embodiments and the accompanying drawings and it should be understood that various changes, substitutions and alterations can be made herein by the skilled in the arts without departing from the sprit and the scope of the present invention as defined by the appended claims.

1. A bidirectional monitoring system capable of a medical diagnosis and a commercial broadcast, comprising:
a main server for providing commercial information, detailed commercial information in case a certain user selects the commercial information, a service page for receiving a user's request for the commercial information, and offering diagnosis result data in response to a medical diagnosis request which is received;
a data communication network for allowing many and unspecified persons to access the main server in an on-line manner; and
a monitoring device for receiving the commercial information from the main server to display them on a main screen after getting an on-line access to the main server via the data communication network, creating a subsidiary window on upper portion of one side of the main screen to output another commercial information or display medical diagnosis request data input according to the user's handling and then transmit them to the main server, and outputting a page for outputting diagnosis result data corresponding to the medical diagnosis request data.

2. The bidirectional monitoring system capable of a medical diagnosis and a commercial broadcast according to claim 1, wherein
the bidirectional monitoring system further comprises a medical institution server, and
the monitoring device includes a connection terminal which can be connected to a health check device, whereby the monitoring device determines if the health check data are input from the health check device when the health check device is connected to the connection terminal, transmits the health check data to the medical institution server in case the health check data are input, compares the health check data with health reference data registered previously so as to output user's health result data recognizable from the health check data on the main screen or the subsidiary window in a graphical illustration, compares the health check data with transmission limitation data registered previously so as to transmit the health check data to the main server or the medical institution server in case the health check data excess the transmission limitation data.

3. The bidirectional monitoring system capable of a medical diagnosis and a commercial broadcast according to claim 2, wherein the health check device includes a blood glucose tester, a blood pressure tester, or a clinical thermometer.

4. The bidirectional monitoring system capable of a medical diagnosis and a commercial broadcast according to claim 2, wherein, in the main server, the commercial information contains link information which enables an on-line connection to a corresponding sponsor's terminal so that an ordering data corresponding to the commercial information can be input by way of an output screen of the commercial information and transmitted.

5. The bidirectional monitoring system capable of a medical diagnosis and a commercial broadcast according to claim 1, wherein
the bidirectional monitoring system further comprises a bidet including a pressure sensor,
the bidet is connected to the monitoring system having functions of a medical diagnosis and a communication, and
the monitoring device is turned on by the pressure sensor in the bidet in case a user sits down on the bidet to accomplish the functions of a medical diagnosis and a communication.

6. The bidirectional monitoring system capable of a medical diagnosis and a commercial broadcast according to claim 1, wherein
the bidirectional monitoring system further comprises an analysis means for collecting user's urine and analyzing the user's urinary composition, and
the monitoring device analyzes the user's health conditions by using the user's urinary composition input from the analysis means, outputs analysis results in a graphical illustration, compares the analysis results with transmission limitation data registered previously, and transmit the analysis results by using data of the user's terminal registered previously in case the analysis results excess the transmission limitation data.

7. The bidirectional monitoring system capable of a medical diagnosis and a commercial broadcast according to claim 6, wherein the monitoring device comprises:
a storage unit for storing user's personal data, mobile terminal numbers corresponding to the user's personal data, and at least one of transmission limitation data and reference data for the urinary composition;
a control unit for outputting the urinary composition data input from the analysis means in a graphical illustration to compare them with the reference data, simultaneously outputting analysis results recognizable from the graphical illustration, and transmitting analysis result data to a corresponding mobile phone to notify it of a recipient in case the analysis result data excess the transmission limitation data;
a display unit for displaying an analysis graph and the analysis results on a screen to show them to the user in response to controlling of the control unit; and
a transmit unit for transmitting the analysis result data to a corresponding mobile phone in response to controlling of the control unit.

8. The bidirectional monitoring system capable of a medical diagnosis and a commercial broadcast according to claim 7, wherein
the monitoring device further comprises a tuner capable of receiving terrestrial broadcast signals, and
the control unit controls the tuner according to predetermined conditions so that broadcast signals of a predetermined channel can be received and displayed on the display unit.

9. The bidirectional monitoring system capable of a medical diagnosis and a commercial broadcast according to claim 7, wherein
the monitoring device further comprises a receive device capable of receiving cable broadcast signals, and
the control unit controls the receive device according to predetermined conditions so that broadcast signals of a predetermined channel can be received and displayed on the display unit.

10. The bidirectional monitoring system capable of a medical diagnosis and a commercial broadcast according to claim 8, wherein
the monitoring device further comprises a channel selection button to allow the user to select a certain channel, and
the control unit controls the tuner or the receive device in response to the channel selection signals selected by the channel selection button.

11. The bidirectional monitoring system capable of a medical diagnosis and a commercial broadcast according to claim 7, wherein the control unit performs controlling in such a way that the screen on the display unit can be split into a plurality of windows and different image signals are output to the split windows.

12. The bidirectional monitoring system capable of a medical diagnosis and a commercial broadcast according to claim 11, wherein broadcast signals output to the split window correspond to commercial broadcast signals.

13. The bidirectional monitoring system capable of a medical diagnosis and a commercial broadcast according to claim 7, wherein the monitoring device further comprises a protective plate for protecting the display unit.

14. The bidirectional monitoring system capable of a medical diagnosis and a commercial broadcast according to claim 7, wherein
the monitoring device further comprises an attack detect sensor, and
the control unit performs controlling in such a way that the an alarm sound can be output to a speaker in response to an attack detect signal from the attack detect sensor.

15. The bidirectional monitoring system capable of a medical diagnosis and a commercial broadcast according to claim 6, wherein the monitoring device accumulates/stores the analysis results input from the analysis means according to respective users and then transmits them on a regular or irregular basis by using data of user's terminals registered according to users.

16. The bidirectional monitoring system capable of a medical diagnosis and a commercial broadcast according to claim 6, wherein the data of user's terminals registered previously in the monitoring device are stored in relation with respective user's personal data and correspond to terminal numbers for a connection to the doctor in charge.

17. The bidirectional monitoring system capable of a medical diagnosis and a commercial broadcast according to claim 7, wherein the transmit unit transmits the analysis results in a manner that a recipient receives them by means of a literal message.

18. The bidirectional monitoring system capable of a medical diagnosis and a commercial broadcast according to claim 7, wherein the monitoring device further comprises a personal identification data input unit through which a certain user can input personal data.

19. The bidirectional monitoring system capable of a medical diagnosis and a commercial broadcast according to claim 18, wherein the personal identification data input unit includes at least one of a key panel, a card reader, and a fingerprint detector.

20. The bidirectional monitoring system capable of a medical diagnosis and a commercial broadcast according to claim 6, wherein the analysis means comprises:
a urine detect sensor for sampling a small amount of user's urine to detect it;
an analyzer for absorbing a small amount of the urine from the urine detect sensor and analyzing its urinary composition; and
a communication interface unit for converting the urinary composition analyzed by the analyzer into urinary composition data and transmitting them to a liquid crystal display means.

21. The bidirectional monitoring system capable of a medical diagnosis and a commercial broadcast according to claim 20, wherein the analysis means further comprises a urine test paper or a urine detect sensor in lower end of the analyzer in order to absorb a small amount of the user's urine.

22. The bidirectional monitoring system capable of a medical diagnosis and a commercial broadcast according to claim 20, wherein the urine test paper can be replaced with new one once it is used, and the urine detect sensor can be replaced with new one after a predetermined period of usage.

23. The bidirectional monitoring system capable of a medical diagnosis and a commercial broadcast according to claim 6, wherein the analysis means comprises:
a sense unit for sensing when a user urinates and outputting a urination sense signal;
a control unit for controlling the whole system in such a way that the user's urine can be analyzed in response to the urination sense signal;
a reservoir open/close valve for closing a reservoir in response to controlling of the control unit at an initial time of the urinary analysis to collect a predetermined amount of user's urine and opening the reservoir in response to controlling of the control unit at an end of the urinary analysis to discharge the user's urine;
a cleaning water supply valve for injecting cleaning water in a water tank into the inside of the reservoir where urine has been discharged in response to controlling of the control unit to wash urinary remnants out;
an analyzer for absorbing a small amount of the user's urine collected in the reservoir and analyzing them by using an analysis program to transmit its analysis results to the control unit; and
a communication interface unit for converting urinary composition analyzed by the analyzer into urinary composition data and transmitting them to the liquid crystal display means.

24. The bidirectional monitoring system capable of a medical diagnosis and a commercial broadcast according to claim 9, wherein
the monitoring device further comprises a channel selection button to allow the user to select a certain channel, and
the control unit controls the tuner or the receive device in response to the channel selection signals selected by the channel selection button.
